(19) United States
(12) Patent Application Publication
(10) Pub. No.: US 2026/0314953 A1
(43) Pub. Date: Oct. 15, 2026

(54) PATIENT SUPPORT APPARATUS WITH GRAPHICAL GUIDANCE

(71) Applicant: Stryker Corporation, Kalamazoo, MI (US)

(72) Inventors: Sujay Sukumaran, Portage, MI (US); Madhu Thomas, London, Ontario (CA); Berkay Güncan, Talas, Kayseri (TR); Christian Fulljames, Grand Rapids, MI (US); Daniel Dehoog, Grand Rapids, MI (US); Curt Stienstra, Lowell, MI (US)

(21) Appl. No.: 18/573,614

(22) PCT Filed: Oct. 7, 2022
(86) PCT No.: PCT/US2022/046081

§ 371 Date: Dec. 22, 2023

(51) Int. Cl. A61B 5/11; A61B 5/00

(52) U.S. Cl. CPC A61B 5/1115; A61B 5/6891; A61B 2560/0223; A61B 2562/0219

(57) ABSTRACT

A patient support apparatus, such as a bed, cot, stretcher, etc., includes a plurality of force sensors that may be used for implementing a scale function and/or an exit detection function. A controller onboard the patient support apparatus is adapted to determine if a patient's center of gravity is within a zone as a prerequisite for carrying out the exit detection function. If the patient's center of gravity is outside the zone, the controller provides graphical guidance to the user regarding the patient's current center of gravity and a target location where the patient's center of gravity should be. The controller may also provide graphical guidance for a user to implement a calibration process for the force sensors, and in some case, one or more accelerometers. The controller may also simultaneously display successive calculations of the patient's center of gravity with a directional indicator showing a direction of its movement.

### BACKGROUND

The present disclosure relates to patient support apparatuses, such as beds, cots, stretchers, recliners, or the like. More specifically, the present disclosure relates to patient support apparatuses that include a plurality of force sensors.

Existing hospital beds and/or stretchers often include a plurality of force sensors that may be used for implementing a scale function and/or an exit detection function. The scale function is used for weighing the patient and/or measuring the weight of other objects on the patient support apparatus. The exit detection function is used for issuing an alarm when the patient exits the patient support apparatus. In order for either of these functions to operate properly, the force sensors have to be calibrated. In addition, in many exit detection system, the patient has to be in a particular position before the exit detection function can be successfully implemented.

### SUMMARY

According to various embodiments, an improved patient support apparatus is provided that helps users execute the scale and/or exit detection function. In some aspects, the patient support apparatus provides graphical guidance to the user for performing a calibration process for the force sensors. Alternatively, or additionally, the patient support apparatus provides graphical indications to the user during an arming step of the exit detection function, thereby enabling users to more easily fix incorrect patient positions. Still further, in some aspects, the movement of the patient's center of gravity may be displayed through a plurality of icons shown in a display that correspond to successive readings of the patient's center of gravity. Still other features and aspects of the present disclosure will be apparent to those skilled in the art in light of the accompanying drawing and the following written description.

According to a first aspect of the present disclosure, a patient support apparatus is provided that includes a support surface, a plurality of force sensors, a control panel, a display, and a controller. The support surface is adapted to support a patient thereon. The plurality of force sensors are adapted to detect downward forces exerted on the support surface. The control panel includes an exit detection arming control adapted to arm an exit detection function. The controller is adapted to calculate a center of gravity of the patient and, in response to a caregiver activating the exit detection arming control, determine whether the patient's center of gravity is inside or outside of a zone. If the controller determines that the patient's center of gravity is outside of the zone, the controller is further adapted to perform the following: (1) display a patient support apparatus icon; (2) display a first indicator at a first location on the patient support apparatus icon, the first location corresponding to the patient's center of gravity; and (3) display a target on the patient support apparatus icon, the target indicating where the patient's center of gravity should be in order for the controller to successfully arm the exit detection function.

According to another aspect of the present disclosure, a patient support apparatus is provided that includes a support surface, a plurality of force sensors, a control panel, a display, and a controller. The support surface is adapted to support a patient thereon. The plurality of force sensors are adapted to detect downward forces exerted on the support surface. The control panel includes a control adapted to control a function that utilizes outputs from the plurality of force sensors. The controller is adapted to display a calibration screen on the display that is adapted to guide a user through a calibration process for calibrating the plurality of force sensors. The calibration screen includes a patient support apparatus icon and a first location icon on the patient support apparatus icon indicating a first calibration location where the user should place a known weight on the patient support apparatus. The controller is adapted to provide a first notification on the display after the user has placed the known weight on the patient support apparatus at the first calibration location and the controller has taken a first set of readings from the plurality of force sensors.

According to another aspect of the present disclosure, a patient support apparatus is provided that includes a support surface, a plurality of force sensors, a control panel, a display, and a control. The support surface is adapted to support a patient thereon. The plurality of force sensors are adapted to detect downward forces exerted on the support surface. The control panel includes a control adapted to control a function that utilizes outputs from the plurality of force sensors. The controller is adapted to repetitively calculate the patient's center of gravity using outputs from the plurality of force sensors and to simultaneously display a plurality of icons on the display. The plurality of icons indicate the locations of successive calculations of the patient's center of gravity.

According to other aspects of the present disclosure, the controller is further adapted to display an arrow on the display indicating a direction in which the patient's center of gravity should move to get to the target.

In some aspects, the first location corresponds to the patient's center of gravity at a time when the caregiver activates the exit detection arming control, and the controller is further adapted to display a second indicator at a second location on the patient support apparatus icon. The second location corresponds to the patient's current center of gravity.

The controller, in some aspects, is further adapted to update the second location as the patient's center of gravity moves.

The target, in some aspects, is displayed as a rectangle icon on the patient support apparatus that corresponds to the zone in which the patient's center of gravity must be located in order to arm the exit detection function.

In some aspects, the controller is further adapted to change a characteristic of the rectangle icon when the patient's current center of gravity moves inside the zone. The changed characteristic may be a color of the rectangle icon.

The controller, in some aspects, is adapted to arm the exit detection function after the patient's center of gravity moves inside the zone.

In some aspects, after arming the exit detection, the controller is further adapted to repetitively calculate the patient's center of gravity and to display a plurality of icons on the patient support apparatus icon. The plurality of icons indicate the locations of successive calculations of the patient's center of gravity.

In some aspects, the plurality of icons includes at least three icons displayed simultaneously on the display.

In some aspects, the controller is further adapted to display a direction in which the successive calculations of the patient's center of gravity are moving.

The controller, in some aspects, is further adapted to display a calibration screen on the display that is adapted to guide a user through a calibration process for calibrating the plurality of force sensors.

The calibration screen, in some aspects, includes a first icon indicating a calibration location where the user should place a known weight on the patient support apparatus.

The controller, in some aspects, is further adapted to change a characteristic of the first icon after the user has placed the known weight on the patient support apparatus at the calibration location and the controller has taken a set of readings from the plurality of force sensors.

In some aspects, the calibration screen includes a plurality of location icons indicating a plurality of calibration locations where the user should place a known weight on the patient support apparatus.

The controller, in some aspects, is further adapted to display a notification on the display indicating which of the plurality of calibration locations the user has placed the known weight.

In some aspects, the controller is adapted to change a color of each of the plurality of location icons after the user has placed the known weight at the corresponding calibration location.

In some aspects, the controller is further adapted to display a second location icon on the patient support apparatus icon indicating a second calibration location where the user should place a known weight on the patient support apparatus. The controller is further adapted to provide a second notification on the display after user has placed the known weight on the patient support apparatus at the second calibration location and the controller has taken a second set of readings from the plurality of force sensors.

In some aspects, the controller is further adapted to display a third location icon on the patient support apparatus icon indicating a third calibration location where the user should place a known weight on the patient support apparatus. The controller is further adapted to provide a third notification on the display after user has placed the known weight on the patient support apparatus at the third calibration location and the controller has taken a third set of readings from the plurality of force sensors.

The controller, in some aspects, is adapted to use the first, second, and third sets of readings to calibrate the plurality of force sensors.

In some aspects, the first notification includes changing a color of the first location icon, the second notification includes changing a color of the second location icon, and the third notification includes changing a color of the third location icon.

The patient support apparatus, in some aspects, further includes a litter frame adapted to support the support surface, as well as an accelerometer adapted to measure a tilt of the litter frame. The controller may be further adapted to also calibrate the accelerometer during the force sensor calibration process.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to one embodiment of the disclosure;

FIG. 2 is a perspective view of a litter frame of the patient support apparatus;

FIG. 3 is a perspective view of a base of the patient support apparatus;

FIG. 4 is a plan view of a control panel that may be included with the patient support apparatus;

FIG. 5 is a block diagram of a control system of the patient support apparatus, as well as several external devices with which the patient support apparatus may be configured to communicate;

FIG. 6 is an illustrative menu screen displayable on the control panel of FIG. 4 illustrating a plurality of control options;

FIG. 7 is an illustrative service screen displayable in response to a user selecting the “service” option on the menu screen of FIG. 6;

FIG. 8 is an illustrative configuration screen displayable in response to a user selecting the “configuration” option on the service screen of FIG. 7;

FIG. 9 is a first scale calibration screen displayable in response to a user selecting the “scale calibration” option on the configuration screen of FIG. 8;

FIG. 10 is a second scale calibration screen in response to a user selecting the “start configuration” option on the scale calibration screen of FIG. 9;

FIG. 11 is a third scale calibration screen displayable in response to a user successfully entering a PIN on the screen of FIG. 10;

FIG. 12 is a fourth scale calibration screen displayable in response to a user selecting the “continue” option on the screen of FIG. 11;

FIG. 13 is a fifth scale calibration screen displayable in response to a user selecting the “continue” option on the screen of FIG. 12;

FIG. 14 is a sixth scale calibration screen displayable in response to a user selecting the “continue” option on the screen of FIG. 13;

FIG. 15 is a seventh scale calibration screen displayable in response to a user selecting the “continue” option on the screen of FIG. 14;

FIG. 16 is a first example of an exit detection error screen displayable in response to a user attempting to arm the exit detection function while the patient's center of gravity is in an incorrect position;

FIG. 17 is a second example of an exit detection error screen displayable in response to a user attempting to arm the exit detection function while the patient's center of gravity is in an incorrect position;

FIG. 18 is an exit detection information screen that illustrates information about the exit detection function, including a series of successive readings of the patient's center of gravity;

FIG. 19 is patient location screen that illustrates information about a series of successive readings of the patient's center of gravity;

FIG. 20 is an exit detection zone customization screen that allows a user to customize a size for an exit detection zone; and

FIG. 21 is an exit detection zone selection screen that allows a user to select predefined exit detection zones according to different patient support apparatus configurations.

### DETAILED DESCRIPTION OF THE EMBODIMENTS

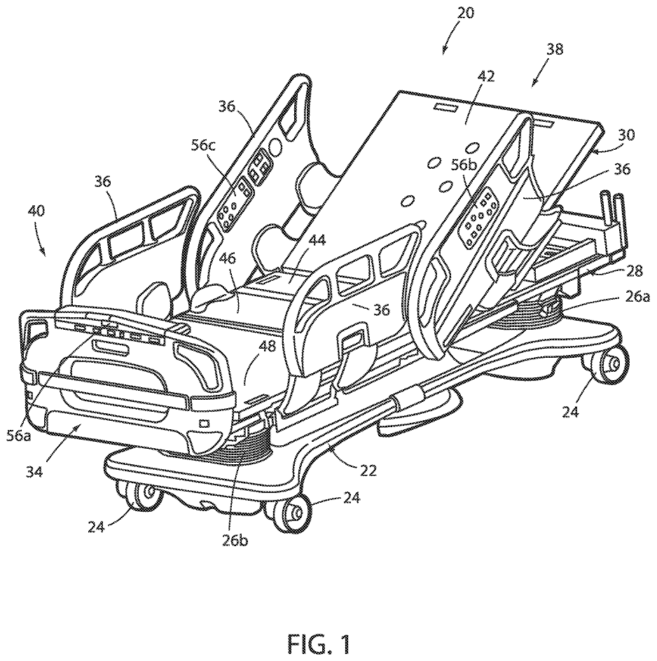

An illustrative patient support apparatus 20 that may incorporate one or more aspects of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a gurney, a recliner, a residential bed, or any other structure capable of supporting a patient, whether stationary or mobile and/or whether medical or residential.

In general, patient support apparatus 20 includes a base 22 having a plurality of wheels 24, a pair of lifts 26 supported on the base, a litter frame 28 supported on the lifts 26, and a support deck 30 supported on the litter frame 28. Patient support apparatus 20 further includes a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, footboard 34, and siderails 36. Support deck 30 provides a support surface for a mattress (not shown), or other soft cushion, so that a person may lie and/or sit thereon. Support deck 30 is made of a plurality of sections, some of which are pivotable about generally horizontal pivot axes. In the embodiment shown in FIG. 1, support deck 30 includes at least a head section 42, a seat section 44, a thigh section 46, and a foot section 48, all of which generally form flat surfaces for supporting the mattress. Head section 42, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width. Notionally, the first standard width may be considered a 36 inch width, the second intermediate width may be considered a 42 inch width and the third more expanded width may be considered a 48 inch width, although these numerical widths may be varied to comprise different width values.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

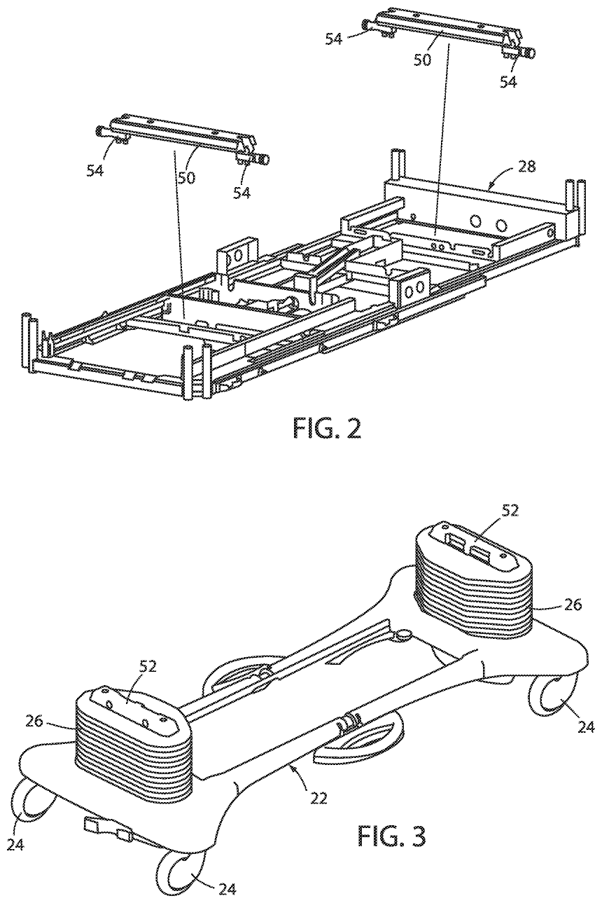

FIG. 2 illustrates in greater detail litter frame 28 separated from lifts 26 and base 22. Litter frame 28 is also shown in FIG. 2 with support deck 30 removed. Litter frame 28 is supported by two lift header assemblies 50. A first one of the lift header assemblies 50 is coupled to a top 52 (FIG. 3) of a first one of the lifts 26, and a second one of the lift header assemblies 50 is coupled to the top 52 of the second one of the lifts 26. Each lift header assembly 50 includes a pair of force sensors 54, which may be load cells, or other types of force sensors. The illustrated embodiment of patient support apparatus 20 therefore includes a total of four force sensors 54, although it will be understood by those skilled in the art that different numbers of force sensors may be used in accordance with the principles of the present disclosure. Force sensors 54 are configured to support litter frame 28. More specifically, force sensors 54 are configured such that they provide complete and exclusive mechanical support for litter frame 28 and all of the components that are supported on litter frame 28 (e.g. support deck 30, footboard 34, the headboard, siderails 36, etc.). Because of this construction, force sensors 54 are adapted to detect the weight of not only those components of patient support apparatus 20 that are supported by litter frame 28 (including litter frame 28 itself), but also any objects or persons who are wholly or partially being supported by support deck 30. The outputs of force sensors 54 are used for implementing a scale function and an exit detection function, as will be described in greater detail below.

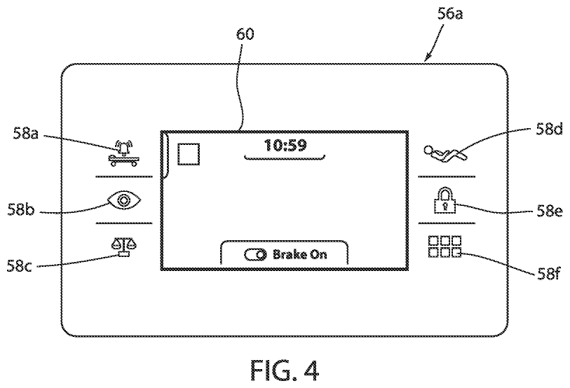

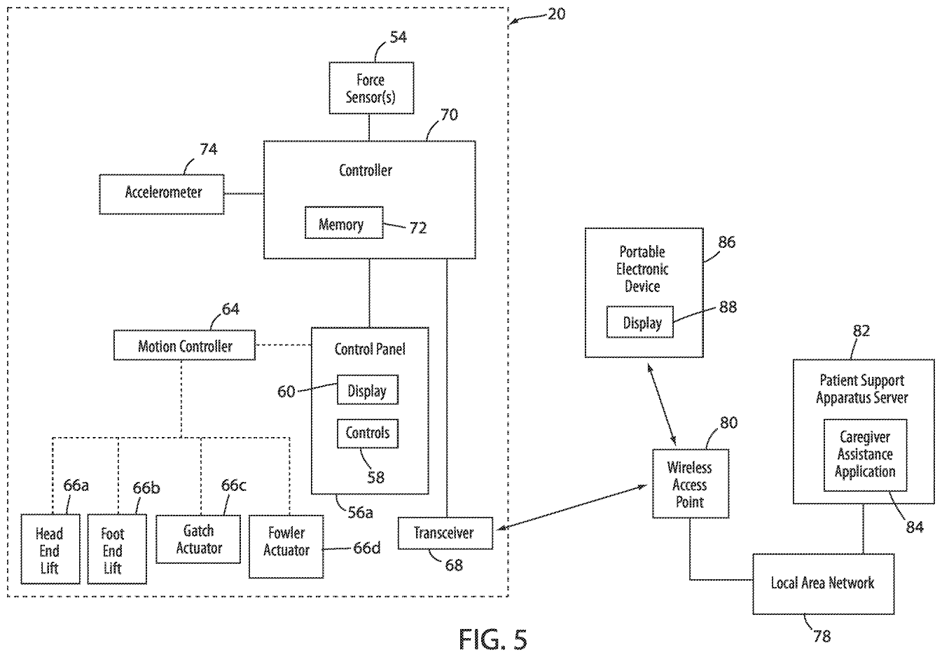

Patient support apparatus 20 further includes a plurality of control panels 56 (FIG. 1) that enable a user of patient support apparatus 20, such as a patient and/or an associated caregiver, to control one or more aspects of patient support apparatus 20. In the embodiment shown in FIG. 1, patient support apparatus 20 includes a footboard control panel 56a, a pair of outer siderail control panels 56b (only one of which is visible), and a pair of inner siderail control panels 56c (only one of which is visible). Footboard control panel 56a concealed by a lid in FIG. 1, but is otherwise visible and accessible to a caregiver when the lid is lifted. In some embodiments, the lid is eliminated from patient support apparatus 20 and the control panel 56a is always visible. Footboard control panel 56a and outer siderail control panels 56b are intended to be used by caregivers, or other authorized personnel, while inner siderail control panels 56c are intended to be used by the patient associated with patient support apparatus 20. Each of the control panels 56 includes a plurality of controls 58 (see, e.g. FIGS. 4-5), although each control panel 56 does not necessarily include the same controls and/or functionality.

Among other functions, controls 58 of control panel 56a allow a user to control one or more of the following: change a height of support deck 30, raise or lower head section 42, take patient weight readings, arm and disarm an exit detection function, activate and deactivate a brake for wheels 24, arm and disarm one or more patient support apparatus monitoring functions, change various settings on patient support apparatus 20, view the current location of the patient support apparatus 20 as determined by a location detection system, perform a calibration process on the patient support apparatus 20, and perform still other actions. One or both of the inner siderail control panels 56c also include at least one control 58 that enables a patient to call a remotely located nurse (or other caregiver).

Control panel 56a includes a display 60 (FIG. 4) configured to display a plurality of different screens thereon. Surrounding display 60 are a plurality of navigation controls 58a-f that, when activated, cause the display 60 to display different screens on display 60. For example, when a user presses navigation control 58a, control panel 56a displays an exit detection control screen on display 60 that includes one or more icons that, when touched, control an onboard exit detection function. The exit detection function is adapted to issue an alert when a patient exits from patient support apparatus 20. Such an exit detection function may include any of the same features and/or functions as, and/or may be constructed in any of the same manners as, the exit detection systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and/or the exit detection system disclosed in commonly assigned U.S. Pat. No. 5,276,432 issued to Travis and entitled PATIENT EXIT DETECTION MECHANISM FOR HOSPITAL BED, the complete disclosures of both of which are incorporated herein by reference.

When a user presses navigation control 58b (FIG. 4), control panel 56a displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 58c, control panel 56a displays a scale control screen that includes a plurality of control icons that, when touched, control the scale system of patient support apparatus 20. In addition to various features of the scale system that are discussed in greater detail below, the scale system of patient support apparatus 20 may include any of the same features, components, and/or and functions as the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system, in some embodiments, or it may utilize one or more different sensors. Further details regarding the scale system are described in greater detail below.

When a user presses navigation control 58d, control panel 56 displays a motion control screen that includes a plurality of control icons that, when touched, control the movement of various components of patient support apparatus 20, such as, but not limited to, the height of litter frame 28 and the pivoting of head section 42. In some embodiments, the motion control screen displayed on display 60 in response to pressing control 58d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 58e (FIG. 4), control panel 56a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

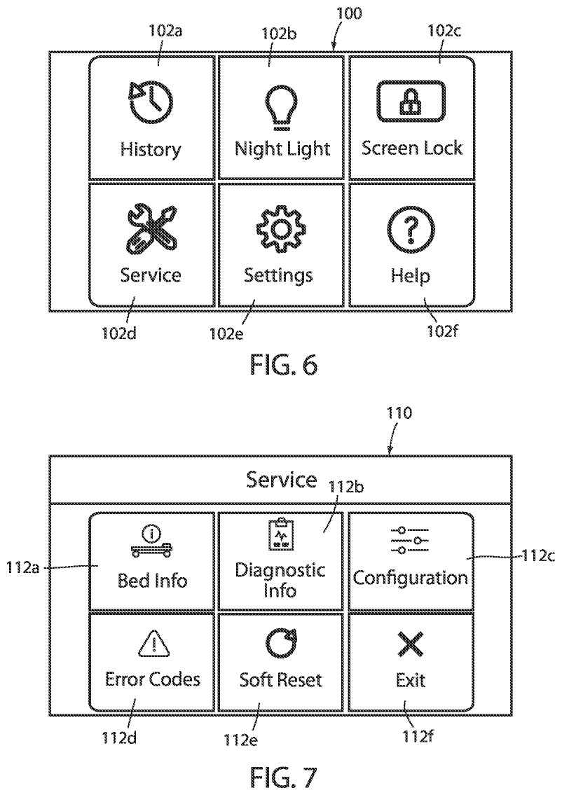

When a user presses on navigation control 58f, control panel 56a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a menu screen in shown herein in FIG. 6. Another example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 58f allows a user to navigate to a screen that enables a user to configure the communication settings between patient support apparatus 20 and one or more wall units. Examples of the type of communication settings that may be configured in this manner are disclosed in, and illustrated in FIGS. 9-15 of, commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION, the complete disclosure of which is incorporated herein by reference.

For all of the navigation controls 58a-f (FIG. 4), screens other than the ones specifically mentioned above may be displayed on display 60 in other embodiments of patient support apparatus 20 in response to a user pressing these controls. Thus, it will be understood that the specific screens mentioned above are merely representative of the types of screens that are displayable on display 60 in response to a user pressing on one or more of navigation controls 58a-f. It will also be understood that, although navigation controls 58a-f have all been illustrated in the accompanying drawings as dedicated controls that are positioned adjacent display 60, any one or more of these controls 58a-f could alternatively be touchscreen controls that are displayed at one or more locations on display 60. Still further, although controls 58a-f have been shown herein as buttons, it will be understood that any of controls 58a-f could also, or alternatively, be switches, dials, or other types of non-button controls.

As shown in FIG. 5, patient support apparatus 20 includes a control system 62 that controls various aspects of patient support apparatus 20. Control system 62 includes control panels 56 (only one of which—56a—is shown in FIG. 5), force sensors 54, a motion controller 64, a plurality of motorized actuators 66, a network transceiver 68, one or more accelerometers 74, and a main controller 70. Main controller 70 includes a memory 72. Main controller 70 and force sensors 54 together act to perform both a scale function and an exit detection function. When carrying out the exit detection function, main controller 70 and force sensors 54 are adapted to determine when an occupant leaves patient support apparatus, or moves in a way indicative of an imminent departure from the patient support apparatus. In such situations, main controller 70 is configured to issue an alert and/or notification to appropriate personnel so that proper steps can be taken in response to the occupant's departure, or imminent departure, in a timely fashion. When carrying out the scale function, the outputs of force sensors 54 are summed together to determine a patient's weight (after adjusting for the tare weight and/or other weights that may be stored in an equipment weight log).

Force sensors 54 are adapted to detect downward forces exerted by an occupant of support deck 30. Thus, when an occupant is positioned on support deck 30 and remains substantially still (i.e. not moving in a manner involving accelerations that cause forces to be exerted against support deck 30), force sensors 54 will detect the weight of the occupant (as well as the weight of any components of patient support apparatus 20 that are supported-directly or indirectly-by force sensors 54). In at least one embodiment, force sensors 54 are load cells. However, it will be understood by those skilled in the art, that force sensors 54 may be implemented as other types of sensors, such as, but not limited to, linear variable displacement transducers and/or any one or more capacitive, inductive, and/or resistive transducers that are configured to produce a changing output in response to changes in the force exerted against them.

Main controller 70 and motion controller 64 are constructed of any electrical component, or group of electrical components, that are capable of carrying out the functions described herein. In many embodiments, controllers 64 and 70 are conventional microcontrollers, although not all such embodiments need include a microcontroller. In general, controllers 64 and 70 include any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. Indeed, in some embodiments, main controller 70 and motion controller 64 are combined with each other and/or with other circuitry or controllers that are present on patient support apparatus 20. The instructions followed by controllers 64 and 70 in carrying out the functions described herein, as well as the data necessary for carrying out these functions, are stored in one or more memories that are accessible to them (e.g. memory 72 for main controller 70).

Although patient support apparatus 20 includes a total of four force sensors 54, it will be understood by those skilled in the art that different numbers of force sensors 54 may be used in accordance with the principles of the present disclosure. Force sensors 54, in at least one embodiment, are configured to support litter frame 28. When so configured, force sensors 54 are constructed to provide complete and exclusive mechanical support for litter frame 28 and all of the components that are supported on litter frame 28 (e.g. deck 30, footboard 34, and, in some embodiments, siderails 36). Because of this construction, force sensors 54 are adapted to detect the weight of not only those components of patient support apparatus 20 that are supported by the litter frame 28 (including litter frame 28 itself), but also any objects or persons who are positioned either wholly or partially on support deck 30. By knowing the weight of the components of the patient support apparatus 20 that are supported on litter frame 28, controller 70 is able to determine a tare weight that, when subtracted from a total weight sensed after a patient is supported on support deck 30, yields a patient weight.

In some embodiments, the physical location of the force sensors 54 on patient support apparatus 20 may be modified to be located on the base frame, such as shown in commonly assigned U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. In other embodiments, the physical location of the force sensors 54 on patient support apparatus 20 may be the same as the position of the load cells disclosed in commonly assigned U.S. patent application Ser. No. 15/266,575 filed Sep. 15, 2016, by inventors Anuj Sidhu et al. and entitled PERSON SUPPORT APPARATUSES WITH EXIT DETECTION SYSTEMS, the complete disclosure of which is also incorporated herein by reference. In still other embodiments, the physical location of the force sensors 54 may be the same as the position of the load cells disclosed in U.S. Pat. No. 7,962,981 issued to Lemire et al. and entitled HOSPITAL BED, the complete disclosure of which is also incorporated herein by reference. In still other embodiments, force sensors 54 may be positioned on patient support apparatus 20 at still other locations.

Motion controller 64 (FIG. 5) is adapted to control the movement of a plurality of components of patient support apparatus 20. These components includes, but are not limited to, a head end lift actuator 66a, a foot end lift actuator 66b, a gatch actuator 66c, and a Fowler actuator 66d. Each of these actuators 66a-d may comprise a linear actuator with a motor built therein. In some embodiments, the linear actuator may be of the type disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In other embodiments, other types of powered actuators may be used, such as, but not limited to, hydraulic and/or pneumatic actuators.

In some embodiments, motion controller 64 operates in the same or similar manners to the main microcontroller 58 and its associated circuitry disclosed in commonly assigned U.S. Pat. No. 10,420,687 issued Sep. 24, 2019, to inventors Aaron Furman et al. and entitled BATTERY MANAGEMENT FOR PATIENT SUPPORT APPARATUSES, the complete disclosure of which is incorporated herein by reference. In such embodiments, motion controller 64 controls the sending of pulse width modulated (PWM) signals to the motors contained within actuators 66a-d, thereby controlling both the speed and the direction of movement of these actuators. Motion controller 64 may take on other forms as well.

Motion controller 64 is in communication with control panel 56 and receives signals from control panel 56 indicating when a user wishes to move one or more components of patient support apparatus 20. That is, control panel 56 includes one or more controls 58 that are adapted, when activated, to instruct motion controller 64 to carry out the desired movement of the various movable components of patient support apparatus 20, as well as one or more controls for stopping such motion. Such movement includes, but is not limited to, raising and lowering the height of litter frame 28, pivoting the Fowler section 42 up and down about a generally horizontal axis (extending laterally from one side of the patient support apparatus 20 to the other), and/or lifting and lowering a knee gatch on patient support apparatus 20.

Head end lift actuator 66a is configured to change the height of the head end 38 of litter frame 28. Foot end lift actuator 66b is configured to change the height of the foot end 40 of litter frame 28. When both of these actuators 66a and 66b are operated simultaneously and at the same speed, the height of litter frame 28 is raised or lowered without changing the general orientation of litter frame 28 with respect horizontal. When one or more of these actuators 66a and/or 66b are operated at different times and/or at different speeds, the orientation of litter frame 28 is changed with respect to horizontal. Lift actuators 66a and 66b are therefore able to tilt litter frame 28 to a variety of different orientations, including, but not limited to, a Trendelenburg orientation and a reverse-Trendelenburg orientation.

Gatch actuator 66c is adapted to raise and lower the joint that couples together the thigh section 46 and the foot section 48 of support deck 30, thereby raising and lowering the portion of the support deck 30 that is positioned close to the patient's knees. Fowler actuator 66d is adapted to raise and lower the head section (or Fowler section) 42 of the support deck 30.

Accelerometer(s) 74 are adapted to detect any tilting of litter frame 28 from a horizontal orientation. In particular, at least one accelerometer 74 is adapted to detect when the litter frame 28 tilts in a manner in which its head end 38 is higher than (or lower than) its foot end 40. In some embodiments, one or more accelerometers 74 are used to detect tilting of litter frame 28 in a transverse direction (i.e. from side to side). The outputs from accelerometers 74 are used by controller 70 to adjust the outputs from the force sensors 54, in at least some embodiments. This is done because, in some embodiments, force sensors 54 are only able to accurately detect purely vertical forces, and when litter frame 28 is tilted, it may exert non-vertical force components onto the force sensors 54 that are not otherwise accurately detectable by the force sensors 54.

Control panel 56 (FIG. 4) communicates with main controller 70 and is adapted to allow a user to control the various functions performed by main controller 70, including, but not limited to, the scale function and the exit detection function carried out by main controller 70 (and force sensors 54). The plurality of controls 58 of control panel 56 may be implemented as buttons, dials, switches, icons on a touchscreen, or other devices. Display 60 of control panel 56 may be a touchscreen that displays one or more controls and/or one or more control screens, some of which are discussed in greater detail below. Display 60 may comprise an LED display, OLED display, or another type of display.

Control system 62 may include additional components beyond those shown in FIG. 5. Alternatively, or additionally, one or more of the components shown in FIG. 5 may be omitted from patient support apparatus 20, or replaced by other components. In some embodiments, control system 62 is configured to automatically maintain a weight log of non-patient items added to, and removed from, the patient support apparatus, thereby making it easier for the caregiver to take accurate readings of the patient's weight. In some such embodiments, control system 62 includes any of the additional components and/or functionality of the scale systems disclosed in the following commonly assigned U.S. patent references: U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. patent 11,33,233 issued to Michael Hayes et al. on Jun. 15, 2021, and entitled PATIENT SUPPORT APPARATUS WITH PATIENT INFORMATION SENSORS; and U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG, the complete disclosures of all of which are incorporated herein by reference.

Network transceiver 68 (FIG. 5) is adapted to communicate with a hospital's local area network 78. In some embodiments, network transceiver 68 is a conventional WiFi transceiver (i.e. IEEE 802.11 . . . ) adapted to wirelessly communicate with one or more wireless access points 80 of a hospital's local area network 78. In other embodiments, network transceiver 68 may be a wireless transceiver that uses conventional 5G technology to communicate with network 78, one or more servers hosted thereon, and/or other devices. In some embodiments, network transceiver 68 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In other embodiments, network transceiver 68 may be a conventional Ethernet transceiver electrically coupled to a conventional Ethernet port (i.e. RJ-45 jack, or the like) built into patient support apparatus 20 that allows a conventional Ethernet cable to be coupled to the patient support apparatus 20. In these embodiments, patient support apparatuses 20 may be coupled to the hospital's local area network 78 by a wired connection. In still other embodiments, patient support apparatus 20 may have both wired and wireless transceivers 68. Still further, in some embodiments, transceiver 68 may take on yet a different form (e.g. a wireless ZigBee transceiver, a Bluetooth transceiver, etc.).

Patient support apparatus 20 uses transceiver 68 (FIG. 5), in some embodiments, to communicate with a patient support apparatus server 82. Patient support apparatus server 82 may be adapted to receive status information from patient support apparatuses 20 and distribute that information to one or more other servers and/or other devices coupled to local area network 78. In at least one embodiment, patient support apparatus server 82 includes a caregiver assistance software application 84 that is adapted to communicate information between both patient support apparatuses 20 and one or more portable electronic devices 86. The portable electronic devices 86 includes, but are not limited to, smart phones, tablets, laptops, Computers on Wheels (COWs), and the like. Each portable electronic device 86 includes a display 88 on which various screens may be displayed, including, in some embodiments, portions of one or more of the screens discussed below. In some embodiments, caregiver assistance application 84 allows authorized users to remotely configure and remotely control various aspects of the patient support apparatuses 20 using their portable electronic device 86. Still further, caregiver assistance application 84 may be adapted to display information about the scale system of the patient support apparatuses 20, including any of the information discussed in greater detail below regarding the scale system.

In any of the embodiments disclosed herein, caregiver assistance application 84 may be configured to include any of the same features or functions as—and/or to operate in any of the same manners as—the caregiver assistance software applications described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. Thus, server 82 may be configured to share with one or more electronic devices 86 any of the information shared with the electronic devices disclosed in these aforementioned patent applications. For example, server 82 may be configured to not only share the location of patient support apparatuses 20 (and any devices that may be associated with them) with electronic devices 86, but it may also forward any of the data generated by patient support apparatuses 20 to the electronic devices 86, thereby letting the caregivers associated with these patient support apparatuses 20 know if, for example, the patient has exited patient support apparatus 20, what the patient's current weight is, whether the patient's cumulative weight change has exceeded a threshold or not, etc. Alternatively, or additionally, patient support apparatus server 82 may forward other patient support apparatus status data (e.g. current siderail position, bed exit status, brake status, height status, scale data, etc.) and/or caregiver rounding information (e.g. when the last rounding was performed for a particular patient, when the next rounds are due, etc.), and/or object data from any objects supported on patient support apparatus 20 to one or more electronic devices 86, thereby providing the caregivers associated with the devices 86 a consolidated portal (e.g. a single software application) for sharing this various information.

It will be understood that, although caregiver assistance application 84 is shown in FIG. 5 to be executed solely by server 82, this may be modified so that caregiver assistance application 84 is executed, either partially or wholly, on one or more of the mobile devices 86 as a mobile app that is downloaded to these devices 86. Similarly, caregiver assistance application 84 may be implemented as an app that executes on server 82 in conjunction with another specialized app that is downloaded to each of the mobile electronic devices 86. Still other variations of caregiver assistance application 84 are possible.

In some embodiments, patient support apparatus server 82 is also configured to determine the location of each patient support apparatus 20, or receive the location of each patient support apparatus 20 from the patient support apparatuses 20. In some embodiments, patient support apparatus server 82 determines the room number and/or bay area of each patient support apparatus 20 that is positioned within a room, as well as the location of patient support apparatuses 20 that are positioned outside of a room, such as, those that may be positioned in a hallway, a maintenance area, or some other area. In general, patient support apparatus server 82 may be configured to determine the position of any patient support apparatus 20 by communicating with one or more nearby wall units (not shown). Further details regarding several manners in which patient support apparatus 20 may be constructed in order to carry out such location communication, as well as the construction and/or operation of such wall units, are disclosed in the following commonly assigned U.S. patent applications: Ser. No. 63/245,245 filed Sep. 17, 2021, by inventors Kirby Niehouser et al., and entitled SYSTEM FOR LOCATING PATIENT SUPPORT APPARATUSES; Ser. No. 63/245,289 filed Sep. 17, 2021, by inventors Madhu Sandeep Thota et al. and entitled PATIENT SUPPORT APPARATUS COMMUNICATION AND LOCATION SYSTEM; Ser. No. 63/26,937 filed May 19, 2020, by inventors Alexander Bodurka et al. and entitled PATIENT SUPPORT APPARATUSES WITH HEADWALL COMMUNICATION; and Ser. No. 63/193,778 filed May 27, 2021, by inventors Krishna Bhimavarapu et al. and entitled PATIENT SUPPORT APPARATUS AND HEADWALL UNIT SYNCHING; the complete disclosures of all of which have already been incorporated herein by reference.

It will be understood that the architecture and content of local area network 78 will vary from healthcare facility to healthcare facility, and that FIG. 5 merely shows a generic example of the type of network a healthcare facility may be employ. Typically, one or more additional servers will be hosted on network 78 and one or more of them may be adapted to communicate with patient support apparatus server 82. For example, an electronic health record server will typically be present in any healthcare facility, and in some embodiments discussed herein, it will be in communication with patient support apparatus server 82 in order to receive patient data that is to be recorded in a patient's health record (e.g. weight readings taken from the scales built into patient support apparatuses 20; therapies provided to patients using a powered mattress onboard patient support apparatuses 20, etc.).

FIG. 6 illustrates a menu screen 100 that may be displayed on display 60 by controller 70. In some embodiments, menu screen 100 is displayed in response to a user activating navigation control 58f (FIG. 4). Alternatively, or additionally, menu screen 100 may be displayed in response to other actions by the user. Menu screen 100 includes several options 102, including a history option 102a, a night light option 102b, a screen lock option 102c, a service option 102d, a settings option 102e, and a help option 102f. If the user presses on, or otherwise selects, the history option 102a, controller 70 is configured to display data gathered over prior usage of patient support apparatus 20, such as, but not limited to patient weight readings, actuator usage, etc. If the user presses on the night light option 102b, controller 70 is configured to display one or more controls for controlling an onboard night light (not shown). If the user presses on screen lock 102c, controller 70 is configured to display one or more options for configuring a screen lock function (e.g. a time until the lock is implemented, whether a passcode is required to unlock it, what information is displayed when the screen is locked, etc.).

If the user presses on service option 102d, controller 70 is configured to display a service screen, such as service screen 110 of FIG. 7, which will be discussed in greater detail below. If the user presses on settings option 102e, controller 70 is configured to display a plurality of patient support apparatus settings that can be controlled by the user, such as, but not limited to, alert settings, motion control settings, etc. If the user presses on help option 102f, controller 70 is configured to display a help screen that includes additional information about how to operate patient support apparatus 20.

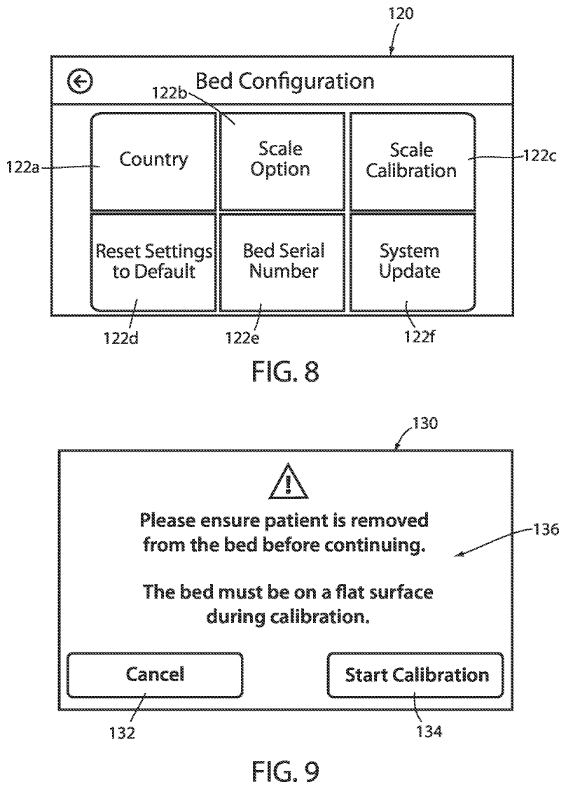

As noted, when a user presses on service option 102d (FIG. 6), controller 70 is configured to display a service screen, such as service screen 110 of FIG. 7. Service screen 110 includes six options: a bed information option 112a, a diagnostic information option 112b, a configuration option 112c, an error code option 112d, a soft reset option 112e, and an exit option 112f. If the user presses on the bed information option 112a, controller 70 is configured to display additional information about the patient support apparatus 20, such as the current software version, date of manufacture, any functional options that are part of patient support apparatus 20, etc. If the user presses on the diagnostic information option 112b, controller 70 is configured to display diagnostic data, including the last service date, a service log, and the like. If the user presses on the configuration option 112c, controller 70 is configured to display a configuration screen, such as the configuration screen 120 of FIG. 8, which will be discussed in greater detail below. If the user presses on the error codes option 112d, controller 70 is configured to display a set of error codes that controller 70 has detected over the course of operation of patient support apparatus 20. If the user presses on soft reset option 112e, controller 70 is configured to turn off and on the patient support apparatus 20, without losing any saved data. If the user presses on the exit option 112f, controller 70 is configured to exit the service screen 110 and return back to menu screen 100, or to a different screen.

As noted, when a user presses on configuration option 112c (FIG. 7), controller 70 is configured to display a configuration screen, such as configuration screen 120 of FIG. 8. Configuration screen 120 includes six options 122: a country option 122a, a scale option 122b, a scale calibration option 122c, a reset option 122d, a serial number option 122e, and a system update option 122f. If the user presses on the country option 122a, controller 70 is configured to present a list of countries to the user which the user can select from. In response to a country selection, controller 70 is configured to change to displaying information on display 60 in the language corresponding to the selected country. If the user presses on the scale option 122b, controller 70 is configured to present one or more options for controlling the onboard scale function, including, in some embodiments, controlling the weight thresholds used for the automatic detection of objects. Several examples of the types of scale options that may be presented to the user in response to pressing scale option 122b are disclosed in commonly assigned U.S. patent application Ser. No. 63/255,223, filed Oct. 13, 2021, by inventors Sujay Sukumaran et al., and entitled PATIENT SUPPORT APPARATUS WITH PATIENT WEIGHT MONITORING, the complete disclosure of which is incorporated herein by reference.

If the user presses on scale calibration option 122c, controller 70 is configured to display a calibration screen, such as first calibration screen 130 of FIG. 9, which will be discussed in greater detail below. If the user presses on the reset option 122d, controller 70 is configured to reset all of the saved settings onboard patient support apparatus 20 to their default parameters. If the user presses on serial number option 122e, controller 70 is configured to display the serial number of that particular patient support apparatus 20. If the user presses on system update option 122f, controller 70 is configured to present a system update screen that is adapted to allow a user to install one or more software updates for patient support apparatus 20.

As was noted above, when a user presses on scale calibration option 122c of configuration screen 120 (FIG. 8), controller 70 is configured to display a first scale calibration screen 130 of the type shown in FIG. 9. Scale calibration screen 130 is displayed when the user wishes to calibrate the force sensors 54 which, as noted, may be load cells. The calibration process is typically performed when the patient support apparatus 20 is initially manufactured. It may also be performed thereafter at various intervals, depending upon the operation of the scale system (e.g. if errors are noted). The calibration process, in essence, informs controller 70 of the response of each load cell 54 to weights applied to them. That is, it informs controller 70 of the voltages that are generated by the load cells 54 in response to the weights supported by them. Controller 70 is therefore given sufficient information to convert the voltages outputs from the load cells 54 into weights that are applied to the load cells 54.

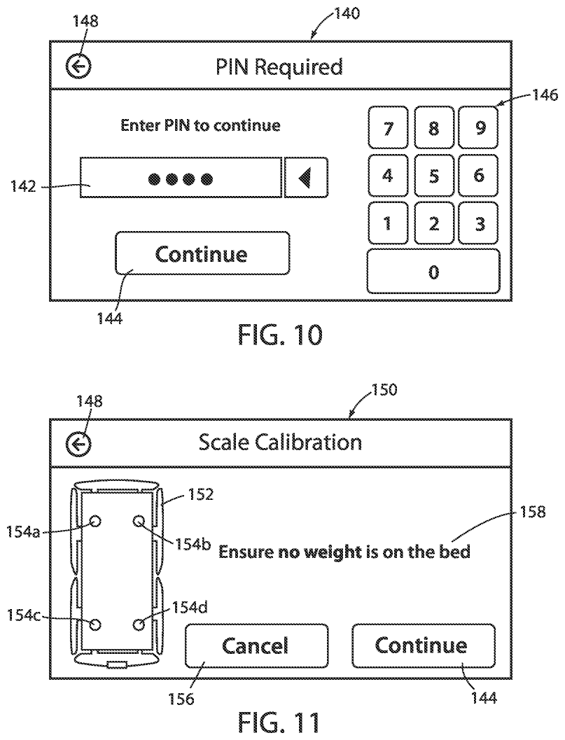

First scale calibration screen 130 (FIG. 9) includes a cancel option 132, a start calibration option 134, and a message 136. Message 136 informs the user that the calibration process requires the patient to be removed from the patient support apparatus 20 and that the patient support apparatus 20 needs to be on a flat surface during the scale calibration process. If the user presses on the cancel option 132, controller 70 cancels the calibration process and returns to displaying the previously displayed screen (e.g. screen 120), or returns to displaying another screen. If the user presses on the start calibration option 134, controller 70 displays a second scale calibration screen 140, such as the second scale calibration screen 140 of FIG. 10.

Second scale calibration screen 140 (FIG. 10) includes a PIN entry field 142, a continue control 144, a numeric keypad 146, and a back option 148. If the user presses the back option 148, controller 70 is configured to display the previously displayed screen (e.g. screen 130 of FIG. 9). Second scale calibration screen 140 is designed to prevent unauthorized users from being able to calibrate the scale system of patient support apparatus 20. That is, only users that know a specific PIN (Personal Identification Number) are able to calibrate the scale system of patient support apparatus. The specific PIN is stored in memory 72 of patient support apparatus 20 and only those individuals that know this PIN are able to calibrate the scale system. Thus, in order for a user to calibrate the scale system, he or she must use the numeric keypad 146 to enter the PIN into PIN entry field 142. Once entered therein, the user presses the continue control 144 and controller 70 compares the PIN entered into field 142 to the PIN stored in memory 72. If there is a match, controller 70 proceeds to display a third scale calibration screen, such as third calibration screen 150 of FIG. 11. If there is no match, controller 70 may return to displaying a previously displayed screen, or alternatively give the user another chance to enter the correct pin by re-displaying screen 140 with a message indicating an incorrect PIN was entered. In some embodiments, controller 70 is configured to only allow the user a limited number of chances to enter the correct PIN before locking out the scale calibration option for a set period of time.

Third scale calibration screen 150 (FIG. 11) includes a patient support apparatus icon 152, a plurality of location icons 154a-d, a cancel control 156, a continue control 144, and a message area 158. Patient support apparatus icon 152 depicts patient support apparatus 20 in a graphical format as viewed from a plan view perspective. The location icons 154a-d are positioned on patient support apparatus icon 152 at locations that correspond to the actual locations of force sensors 54 on patient support apparatus 20. That is, first location 152a is positioned directly above the location of a first force sensor 54 on patient support apparatus 20; second location 152b is positioned directly above the location of a second force sensor 54 on patient support apparatus 20; third location sensor 152c is positioned directly above the location of a third force sensor 54; and fourth location 152d is positioned directly above the location of a fourth force sensor 54.

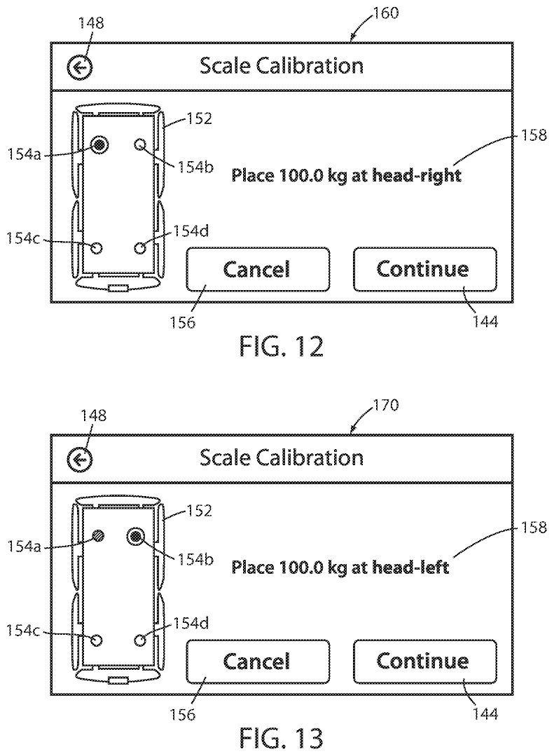

Message area 158 of third calibration screen 150 (FIG. 11) includes a message instructing the user to ensure that no weight is currently on patient support apparatus 20. If the user presses the cancel control 156, controller 70 cancels the calibration process and switches to displaying a non-scale calibration screen, such as, but not limited to, configuration screen 120 (FIG. 8). If the user presses the continue control 144, controller 70 continues with the scale calibration process and switches to displaying a fourth scale calibration screen, such as the fourth scale calibration screen 160 of FIG. 12.

Fourth calibration screen 160 (FIG. 12) includes patient support apparatus icon 152, location icons 154a-d, message area 158, cancel control 156, and continue control 144. Message area 158 displays a message indicating that the user should place a known weight at a right head end location on patient support apparatus 20 that corresponds to the location of first location icon 154a. In order to further clarify the location at which this known weight should be placed, controller 70 has changed the size and color of first location icon 154a. In the example of FIG. 12, controller 70 has changed first location icon 154a to a different color (e.g. from gray to blue) and circled the icon 154a with a circle of a different color (e.g. white). It will be understood that controller 70 may utilize different color changes and/or use different manners of highlighting first location icon 154a to the user so that the user is provided with a visual indication of which location to put the known weight at.

In the example of FIG. 12, controller 70 displays instructions in message area 158 instructing the user to place a weight of 100 kilograms at the right head end location of patient support apparatus 20 that corresponds to location icon 154a. It will be understood that the selection of 100 kilograms is merely one example of the known weights that may be used for the calibration process. In other embodiments, controller 70 may be configured to use a fifty kilogram weight during the calibration process, or any other value of weight ranging from about ten kilograms up to 100 kilograms or more. Whatever the weight that is used, controller 70 is programmed to know that weight and to display it as part of the instructions 158 of FIGS. 12-15. If the user presses the cancel control 156, controller 70 cancels the calibration process and switches to displaying a non-scale calibration screen, such as, but not limited to, configuration screen 120 (FIG. 8). If the user presses the continue control 144, controller 70 continues with the scale calibration process and takes a first set of weight readings from each of the force sensors 54. In addition, controller 70 switches to displaying a fifth scale calibration screen, such as the fifth scale calibration screen 170 of FIG. 13.

The first set of weight readings that are taken prior to displaying the fifth scale calibration screen 170 are stored in memory 72. This first set of weight readings includes a weight reading for each of the four force sensors 54. These four weight readings are taken when the known weight is positioned on patient support apparatus 20 at the location designated by first location icon 154a. The four weight readings of this first set of weight readings are taken at the same time and, as will be discussed in greater detail below, are used with other sets of weight readings to solve a set of four equations having four unknown variables.

Fifth scale calibration screen 170 (FIG. 13) includes patient support apparatus icon 152, location icons 154a-d, message area 158, cancel control 156, and continue control 144. Message area 158 displays a message indicating that the user should move the known weight to a left head end location on patient support apparatus 20 that corresponds to the location of second location icon 154b. In order to further clarify the location at which this known weight should be placed, controller 70 has changed the size and color of second location icon 154b in a similar manner to how it changed the icon 154a in FIG. 12 (i.e. it has changed second location icon 154b to a different color (e.g. from gray to blue) and circled the icon 154b with a circle of a different color (e.g. white). It will be understood that controller 70 may utilize different color changes and/or use different manners of highlighting second location icon 154b to the user so that the user is provided with a visual indication of which location to put the known weight at.

In addition to highlighting second location icon 154b, controller 70 has also changed the color of first location icon 154a in screen 170. This color change is implemented to visually convey to the user that the process of placing the known weight at the location on patient support apparatus 20 corresponding to first location icon 154a has been completed. In some embodiments, controller 70 changes the color of first location icon 154a to green, thus signifying that the calibration process for that particular location has been completed. Other color changes and/or size and/or shape changes may, of course, be utilized.

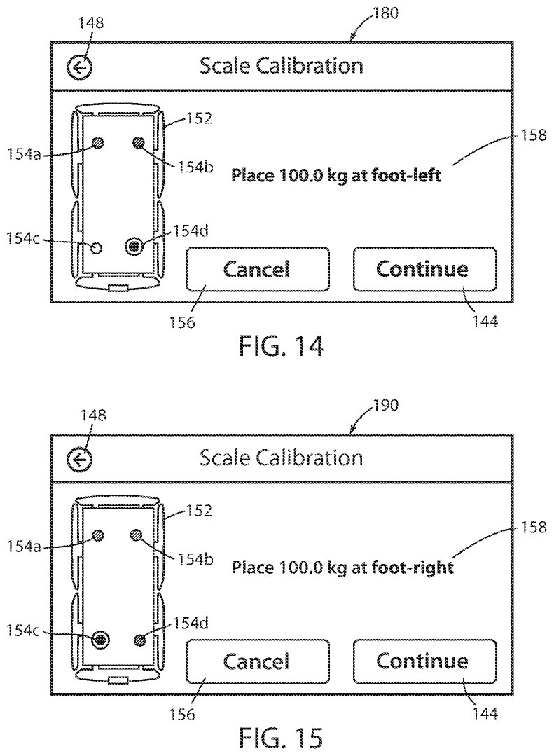

If the user presses the cancel control 156 (FIG. 13), controller 70 cancels the calibration process and switches to displaying a non-scale calibration screen, such as, but not limited to, configuration screen 120 (FIG. 8). If the user presses the continue control 144, controller 70 continues with the scale calibration process and takes a second set of weight readings from each of the force sensors 54. In addition, controller 70 switches to displaying a sixth scale calibration screen, such as the sixth scale calibration screen 180 of FIG. 14.

The second set of weight readings that are taken prior to displaying the sixth scale calibration screen 180 are stored in memory 72. This second set of weight readings includes a weight reading for each of the four force sensors 54. These four weight readings are taken when the known weight is positioned on patient support apparatus 20 at the location designated by second location icon 154b. The four weight readings of this second set of weight readings are taken at the same time and, as will be discussed in greater detail below, are used with other sets of weight readings to solve a set of four equations having four unknown variables.

Sixth scale calibration screen 180 (FIG. 14) includes patient support apparatus icon 152, location icons 154a-d, message area 158, cancel control 156, and continue control 144. Message area 158 displays a message indicating that the user should move the known weight to a left foot end location on patient support apparatus 20 that corresponds to the location of third location icon 154d. In order to further clarify the location at which this known weight should be placed, controller 70 has changed the size and color of third location icon 154d in a manner similar to how it changed the icon 154a in FIG. 12 and the icon 154b in FIG. 13. It will be understood that controller 70 may utilize different color changes and/or use different manners of highlighting third location icon 154d to the user so that the user is provided with a visual indication of which location to put the known weight at.

In addition to highlighting third location icon 154d, controller 70 has also changed the color of first and second location icons 154a and b in screen 180. This color change is implemented to visually convey to the user that the process of placing the known weight at the locations on patient support apparatus 20 corresponding to first and second location icons 154a and b has been completed. In some embodiments, controller 70 changes the color of location icons 154a and 154b to green, thus signifying that the calibration process for these particular locations has been completed. Other color changes and/or size and/or shape changes may, of course be utilized.

If the user presses the cancel control 156 (FIG. 14), controller 70 cancels the calibration process and switches to displaying a non-scale calibration screen, such as, but not limited to, configuration screen 120 (FIG. 8). If the user presses the continue control 144, controller 70 continues with the scale calibration process and takes a third set of weight readings from each of the force sensors 54. In addition, controller 70 switches to displaying a seventh scale calibration screen, such as the seventh scale calibration screen 190 of FIG. 15.

The third set of weight readings that are taken prior to displaying the seventh scale calibration screen 190 are stored in memory 72. This third set of weight readings includes a weight reading for each of the four force sensors 54. These four weight readings are taken when the known weight is positioned on patient support apparatus 20 at the location designated by third location icon 154d. The four weight readings of this third set of weight readings are taken at the same time and, as will be discussed in greater detail below, are used with other sets of weight readings to solve a set of four equations having four unknown variables.

Seventh scale calibration screen 190 (FIG. 15) includes patient support apparatus icon 152, location icons 154a-d, message area 158, cancel control 156, and continue control 144. Message area 158 displays a message indicating that the user should move the known weight to a right foot end location on patient support apparatus 20 that corresponds to the location of fourth location icon 154c. In order to further clarify the location at which this known weight should be placed, controller 70 has changed the size and color of fourth location icon 154c in a manner similar to how it changed the icon 154a in FIG. 12, the icon 154b in FIG. 13, and the icon 154d in FIG. 14. It will be understood that controller 70 may utilize different color changes and/or use different manners of highlighting fourth location icon 154c to the user so that the user is provided with a visual indication of which location to put the known weight at.

In addition to highlighting fourth location icon 154c, controller 70 has also changed the color of first, second, and third location icons 154a, 154b, and 154d in screen 190. This color change is implemented to visually convey to the user that the process of placing the known weight at the locations on patient support apparatus 20 corresponding to first, second, and third location icons 154a, 154b, and 154d has been completed. In some embodiments, controller 70 changes the color of location icons 154a, 154b, and 154d to green, thus signifying that the calibration process for these particular locations has been completed. Other color changes and/or size and/or shape changes may, of course be utilized.

If the user presses the cancel control 156 (FIG. 15), controller 70 cancels the calibration process and switches to displaying a non-scale calibration screen, such as, but not limited to, configuration screen 120 (FIG. 8). If the user presses the continue control 144, controller 70 continues with the scale calibration process and takes a fourth set of weight readings from each of the force sensors 54. In addition, controller 70 switches to displaying a message screen (not shown) that indicates that the calibration was either successfully completed, or that an error has taken place.

The fourth set of weight readings that are taken after the user presses the continue control 144 of FIG. 15 are stored in memory 72. This fourth set of weight readings includes a weight reading for each of the four force sensors 54. These four weight readings are taken when the known weight is positioned on patient support apparatus 20 at the location designated by fourth location icon 154c. The four weight readings of this fourth set of weight readings are taken at the same time and, as will be discussed in greater detail below, are used with the other three sets of weight readings to solve a set of four equations having four unknown variables.

More specifically, controller 70 uses the above-mentioned four sets of weight readings to solve the following four equations for the calibration coefficients C1, C2, C3, and C4:

$$( C {1}^{*} samp\_RH \_LCell1 ) + ( C {2}^{*} samp\_RH \_LCell2 ) + ( C {3}^{*} samp\_RH \_LCell3 ) + ( C {4}^{*} samp\_RH \_LCell4 ) = CalibWeight \qquad ( Equation 1 )$$

$$( C {1}^{*} samp\_LH \_LCell1 ) + ( C {2}^{*} samp\_LH \_LCell2 ) + ( C {3}^{*} samp\_LH \_LCell3 ) + ( C {4}^{*} samp\_LH \_LCell4 ) = CalibWeight \qquad ( Equation 2 )$$

$$( C {1}^{*} samp\_LF \_LCell1 ) + ( C {2}^{*} samp\_LF \_LCell2 ) + ( C {3}^{*} samp\_LF \_LCell3 ) + ( C {4}^{*} samp\_LF \_LCell4 ) = CalibWeight \qquad ( Equation 3 )$$

$$( C {1}^{*} samp\_RF \_LCell1 ) + ( C {2}^{*} samp\_RF \_LCell2 ) + ( C {3}^{*} samp\_RF \_LCell3 ) + ( C {4}^{*} samp\_RF \_LCell4 ) = CalibWeight \qquad ( Equation 4 )$$

The four sample values (samp_RH_LCell1, samp_RH_LCell2, samp_RH_LCell3, and samp_RH_LCell4) of equation 1 correspond to the readings that are taken from each of the four force sensors 54 after the user presses the continue control 144 of calibration screen 160 (FIG. 12). In other words, these four readings are taken when the user has placed the known weight (i.e. CalibWeight=100 kg) on the patient support apparatus 20 at the right head end location corresponding to location icon 154a.

The four sample values (samp_LH_LCell1, samp_LH_LCell2, samp_LH_LCell3, and samp_LH_LCell4) of equation 2 correspond to the readings that are taken from each of the four force sensors 54 after the user presses the continue control 144 of calibration screen 170 (FIG. 13). In other words, these four readings are taken when the user has placed the known weight (i.e. CalibWeight) on the patient support apparatus 20 at the left head end location corresponding to location icon 154b.

The four sample values (samp_LF_Cell1, samp_LF_LCell2, samp_LF_LCell3, and samp_LF_LCell4) of equation 3 correspond to the readings that are taken from each of the four force sensors 54 after the user presses the continue control 144 of calibration screen 180 (FIG. 14). In other words, these four readings are taken when the user has placed the known weight (i.e. CalibWeight) on the patient support apparatus 20 at the left foot end location corresponding to location icon 154d.

The four sample values (samp_RF_Cell1, samp_RF_LCell2, samp_RF_LCell3, and samp_RF_LCell4) of equation 4 correspond to the readings that are taken from each of the four force sensors 54 after the user presses the continue control 144 of calibration screen 190 (FIG. 15). In other words, these four readings are taken when the user has placed the known weight (i.e. CalibWeight) on the patient support apparatus 20 at the right foot end location corresponding to location icon 154c.

In some embodiments, controller 70 is configured to use Cramer's rule to solve these four equations for the constants C1, C2, C3, and C4. After solving for these four constants, controller 70 multiplies the outputs of the corresponding force sensors 54 by the corresponding constant C1, C2, C3, or C4 in order to yield an accurate weight reading from each force sensor 54. In other words, the outputs of the first force sensor 54 are multiplied by C1; the outputs of the second force sensor 54 are multiplied by C2; the outputs of the third force sensor 54 are multiplied by C3; and the outputs from the fourth force sensor 54 are multiplied by C4.

In some embodiments, controller 70 is configured to display one or more additional screens and/or one or more alternative screens during the force sensor calibration process. For example, in some embodiments, controller 70 is configured to display a transition screen when moving from screen 160 to 170, from screen 170 to 180, from screen 180 to 190, and from screen 190 to a subsequent screen. In some embodiments, the transition screen includes a rotating icon, or the like, that indicates that patient support apparatus 20 is working while controller 70 takes each one of the four sets of weight readings. The transition screen may also include a reminder to the user to keep the patient support apparatus 20 free from vibrations and/or other disturbances that would be sensed by the force sensors 54 during this time period. Still other screens may also, or alternatively, be displayed on display 60 by controller 70 during the force sensor calibration process.

In some embodiments, controller 70 may be configured to monitor the outputs of the force sensors 54 (which may be uncalibrated, or whose previous calibration has not yet been updated) to look for disturbances, vibrations, or other unacceptable conditions during the calibration process (e.g. an excessive weight on patient support apparatus 20). If the disturbances or vibrations exceed a preset threshold stored in memory 72, and/or if an excessive total weight is detected, controller 70 is configured to cancel the calibration process (or a portion of the calibration process) and ask that the user re-start the calibration process. In some embodiments, controller 70 is configured to repetitively take weight readings from each of the force sensors 54 during the course of the calibration process and look for readings that are outside of a standard deviation (or some scale factor of the standard deviation). If any readings are found outside of the standard deviation (or the standard deviation scaled by a factor), controller 70 is configured to cancel the calibration process (or the corresponding portion of the calibration process) and inform the user. In this manner, the force sensors 54 are used to confirm that the calibration process takes place without disturbances or vibrations (other than those caused by the repositioning of the known weight at the locations on patient support apparatus 20 corresponding to location icons 154a-d).

In some embodiments, controller 70 is configured to display a confirmation screen or message each time the known weight is placed in one of the four designated locations and controller 70 has successfully taken a weight reading. In other words, controller 70 may be configured to display a pass/fail screen and/or message for each step of the calibration process, thereby providing the user with more immediate feedback of the success or failure of each calibration step.

In some embodiments, controller 70 is configured to also carry out a calibration of the one or more accelerometer(s) 74 onboard patient support apparatus 20 at the same time that the calibration process of force sensors 54 is carried out. In such embodiments, while the user is carrying out the calibration of load cells 54, controller 70 is also taking readings from accelerometer(s) 74 and recording those reading(s). Because patient support apparatus 20 is positioned on a level floor during the calibration of force sensors 54, controller 70 knows that the voltage(s) output from accelerometer(s) 74 during this calibration process should correspond to a tilt angle of litter frame 28 that is zero. Accordingly, controller 70 is configured to use these readings to set the voltage measured from the accelerometer(s) 74 as corresponding to a zero tilt angle. In other words, controller 70 correlate the outputs of accelerometer(s) 74 to the situation where the litter frame has an angular tilt of zero degrees. As was noted, controller 70 is configured, in at least some embodiments, to adjust the outputs of force sensors 54 based on the angular deviation of litter frame 28 from horizontal. Such adjustments are made in a conventional manner using known trigonometric ratios.

Once the force sensors 54 have been calibrated, controller 70 is configured to use the outputs from the force sensors 54 to perform a scale function and/or an exit detection function. When performing the scale function, controller 70 is configured to sum the force detected by the force sensors 54, subtract the tare weight reading (and, in some situations, the weight readings from any objects that are positioned onboard patient support apparatus 20 and whose weight are stored in a weight log), and compute the patient's weight. In some embodiments, the scale functions performed by controller 70 using the outputs from force sensors 54 may include any of the features or functions of the scale systems disclosed in the following commonly assigned U.S. patent references: U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. patent 11,33,233 issued to Michael Hayes et al. on Jun. 15, 2021, and entitled PATIENT SUPPORT APPARATUS WITH PATIENT INFORMATION SENSORS; U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG; and U.S. patent application Ser. No. 63/255,223, filed Oct. 13, 2021, by inventors Sujay Sukumaran et al. and entitled PATIENT SUPPORT APPARATUS WITH PATIENT WEIGHT MONITORING, the complete disclosures of all of which are incorporated herein by reference.

When controller 70 uses the outputs of force sensors 54 to carry out an exit detection function, controller 70 may be configured to implement any of the features, functions, and/or components of any of the exit detection systems disclosed in any of the following commonly assigned U.S. patent references: U.S. patent application Ser. No. 63/245,279 filed Sep. 17, 2021, by inventors Jerald Trepanier et al. and entitled PATIENT SUPPORT APPARATUSES WITH PATIENT MONITORING; U.S. patent application Ser. No. 17/318,476 filed May 12, 2021, by inventors Sujay Sukumaran et al. and entitled PATIENT SUPPORT APPARATUS WITH AUTOMATIC EXIT DETECTION MODES OF OPERATION; U.S. patent application Ser. No. 16/917,004 filed Jun. 30, 2020, by inventors Sujay Sukumaran et al. and entitled PERSON SUPPORT APPARATUS WITH ADJUSTABLE EXIT DETECTION ZONES; and U.S. patent application Ser. No. 15/266,575 filed Sep. 15, 2016, by inventors Anuj K. Sidhu et al. and entitled PERSON SUPPORT APPARATUSES WITH EXIT DETECTION SYSTEMS, the complete disclosures of all of which are incorporated herein by reference.

In some embodiments, in order to instruct controller 70 to carry out the exit detection function, a user first presses on exit detection control 58a (FIG. 4). Activating exit detection control 58a, in some embodiments, causes controller 70 to display an exit detection screen (not shown) that allows a user to select a sensitivity level for the exit detection function. One example of such a screen is shown in FIG. 4 of commonly assigned U.S. patent application Ser. No. 17/318,476 filed May 12, 2021, by inventors Sujay Sukumaran et al. and entitled PATIENT SUPPORT APPARATUS WITH AUTOMATIC EXIT DETECTION MODES OF OPERATION, the complete disclosure of which has already been incorporated herein by reference. In other embodiments, the exit detection function may be implemented with only a single sensitivity, in which case the exit detection screen will eliminate the options for selecting different sensitivity levels. In still other embodiments, when a user activates exit detection control 58a, controller 70 is configured to automatically attempt to arm the exit detection function.

Regardless of the specific controls used, and screens displayed, to activate the exit detection function, once a user has instructed controller 70 to activate the exit detection function, controller 70 begins an arming process that may take several seconds. During this arming process, controller 70 determines a center of gravity of the patient using the outputs from the force sensors 54. Such center of gravity calculations may be performed in a conventional manner. Further details are disclosed in commonly assigned U.S. Pat. No. 5,276,432 issued to Travis and entitled PATIENT EXIT DETECTION MECHANISM FOR HOSPITAL BED, the complete disclosures of which has already been incorporated herein by reference. After determining the patient's center of gravity, controller 70 determines whether the patient's center of gravity is positioned within an arming zone. Further details regarding examples of arming zones are disclosed in the commonly assigned U.S. patent application Ser. No. 17/318,476, mentioned above and incorporated by reference herein. In order for controller 70 to allow the exit detection function to be armed, the patient's center of gravity must be positioned within the arming zone during the arming process.

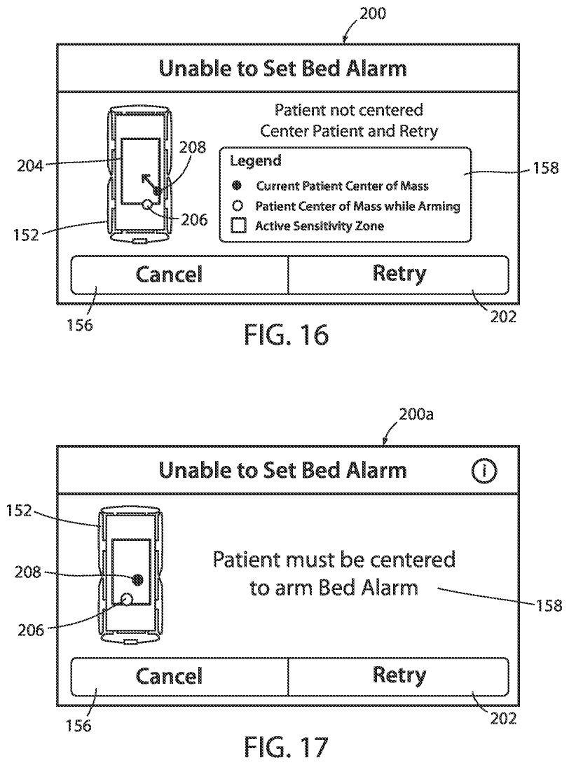

Accordingly, after the user activates a control to arm the exit detection function, controller 70 checks to see if the patient's current center of gravity is positioned inside of an arming zone or not. If it is, controller 70 continues with the arming process until it either arms the exit detection function, or informs the user of another error that prevents successful arming (e.g. excessive vibrations or movement detected, brake not engaged, battery status too low, minimum weight not detected, etc.). If the patient's center of gravity is not inside of the arming zone, controller 70 is configured to display an exit detection error screen, such as the exit detection error screen 200 shown in FIG. 16.

Exit detection error screen 200 (FIG. 16) includes a message area 158, a patient support apparatus icon 152, a cancel control 156, a retry control 202, an arming zone icon 204, an initial patient position icon 206, and a current patient position icon 208. Message area 158 includes a message indicating to the user that the patient's center of gravity was not inside the arming zone during the arming process, and that controller 70 was therefore unable to arm the exit detection function. Message area 158 also includes a legend for the icons 204, 206, and 208.

Arming zone icon 204 is shown as a rectangle in FIG. 16 and corresponds to the arming zone in which the patient's center of gravity must be located during the arming process of the exit detection function. Arming zone icon 204 is positioned on patient support apparatus icon 152 at the same location that the actual arming zone is defined on the actual patient support apparatus 20. Thus, arming zone icon 204 represents a target location for the patient's center of gravity during the exit detection function arming process.

Initial patient position icon 206 indicates the location of the patient's center of gravity as detected during the arming process. In other words, initial patient position icon 206 indicates the patient's center of gravity at the time controller 70 determined it was outside of the arming zone, and thus was unable to complete the exit detection arming process. Current position icon 206 indicates the currently measured position of the patient's center of gravity. Consequently, in the screen 200 shown in FIG. 16, the patient has subsequently moved upward and to the right since the moment the arming process was initiated. Current position icon 208 includes an arrow indicator. This arrow indicator points in a direction in which the patient should be moved in order for the exit detection function to be successfully armed. In some embodiments, the arrow indicator is displayed by controller 70 such that it points toward the center of the arming zone icon 204 (i.e. the center of this rectangle). The arrow indicator provides a graphical indication to the user of which direction the patient needs to be moved in order to complete the arming process.

Controller 70 is configured to repetitively update the current patient position icon 206 as the patient moves so that the caregiver is able to see on screen 200 the patient's current position relative to the arming zone icon 204. This provides visual feedback to the user of their progress, or lack of progress, in getting the patient correctly repositioned. In some embodiments, controller 70 is configured to repetitively update the current patient position icon 206 multiple times a second. In such embodiments, controller 70 takes new readings from each of the force sensors 54 multiple times a second and calculates the patient's center of gravity from those force sensors readings multiple times a second. These calculations are used to update the location of current patient position icon 206 on patient support apparatus icon 152.

If the user presses on the cancel control 156 (FIG. 16), controller 70 is configured to cancel the arming process and to not arm the exit detection function. If the user presses on the retry control 202, controller 70 is configured to re-attempt to arm the exit detection function. Typically, the caregiver will press on the retry control 202 control after he or she has successfully moved the patient's current center of gravity inside the arming zone. The caregiver will know that this has been achieved because screen 200 provides visual feedback in real time to the user of the patient's current position relative to the arming zone. In some embodiments, controller 70 is configured to change an aspect of screen 200 and/or arming zone icon 204 in response to the patient's center of gravity moving inside of the arming zone. For example, in some embodiments, controller 70 is configured to change the color of arming zone icon 204 to green after the patient's current center of gravity is moved to inside the arming zone (i.e. current position icon 208 is moved inside rectangle 204). Additionally, or alternatively, controller 70 may display a message in message area 158 informing the user that the patient's center of gravity is now in an acceptable position. Other changes may also, or alternatively, be implemented, such as, but not limited to, changing the size, color, and/or shape of current position icon 208 (and/or changing other characteristics of arming zone icon 204).

FIG. 17 illustrates an alternative exit detection arming error screen 200a that controller 70 may be configured to display, in an alternative embodiment, in response to the patient being out of position during the exit detection function arming process. Error screen 200a differs from error screen 200 in that it does not include a legend in message area 158, and it illustrates a situation where the patient's current center of gravity has been successfully moved inside the arming zone (e.g. current position icon 208 is positioned inside rectangle icon 204). Further, in this particular example, controller 70 is configured to remove the arrow indicator from current position icon 208 after it has been moved inside of rectangle 204. This provides another indication to the user that the patient's current center of gravity no longer needs to move to another position in order to arm the exit detection function. As noted above, in some embodiments, controller 70 may display arming zone icon 204 in FIG. 17 in a different color than what is shown in FIG. 16 in order to provide the user with more visual guidance that the patient's center of gravity is now in a position to arm the exit detection. Thus, if the user presses the retry control 202 in FIG. 17, controller 70 should allow the exit detection function to be armed, provided the other conditions (i.e. those that are not dependent upon the current patient location) for arming the exit detection function are met.

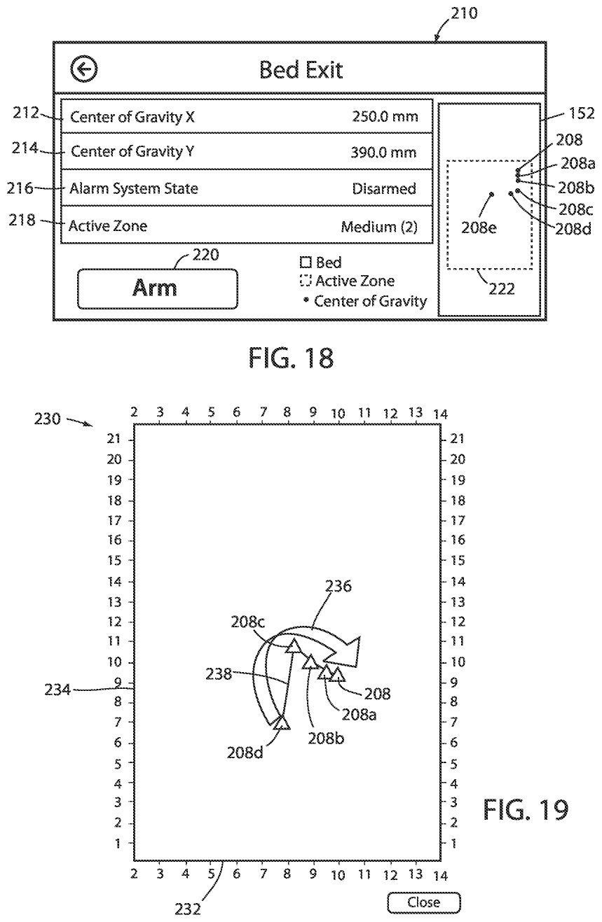

FIG. 18 illustrates an exit detection information screen 210 that may be displayed on display 60 by controller 70 in some embodiments of patient support apparatus 20. Exit detection information screen 210 includes an X indicator 212, a Y indicator 214, an alarm state indicator 216, an active zone indicator 218, an arming control 220, patient support apparatus icon 152, an active zone icon 222, and a plurality of patient position icons 208, 208a-e. X indicator 212 informs the user of the current X position of the patient's center of gravity. The X position, in this particular embodiment, refers to the transverse direction (i.e. from one side of patient support apparatus 20 to the other side). Y indicator 214 informs the user of the current Y position of the patient's center of gravity. The Y position, in this particular embodiment, refers to the longitudinal direction (i.e. from foot end 40 to head end 38). Controller 70 updates X and Y indicators in real time (e.g. multiple times a second).

Alarm state indicator 216 informs the user of the armed/disarmed state of the exit detection function. In other words, armed state indicator 216 lets the user know if the exit detection function is currently armed or currently disarmed. Active zone indicator 218 lets the user know which zone is currently active. Active zone indicator 218 is therefore only present in those patient support apparatus embodiments that have multiple zones for carrying out the exit detection function. As was mentioned previously, the exit detection function may allow the user to select different sensitivity levels (i.e. different amounts of movement necessary to trigger an alarm), and those different sensitivity levels correspond to different active zones (which generally have different sizes, thereby allowing the user different amounts of freedom of movement).

Arming control 220 allows the user to arm the exit detection function when it is activated. When the user activates arming control 220, controller 70 undergoes the arming process described above and, if the patient's center of gravity is located outside of the corresponding arming zone during that process, it displays an error message screen, such as one of those shown in FIG. 16 or 17. Screen 210 (FIG. 18) also displays patient support apparatus icon 152 along with a currently active zone icon 222. Active zone icon 222 is positioned on patient support apparatus icon 152 at the same location that the actual active zone on patient support apparatus 20 is positioned. Further, active zone icon 222 bears the same size and shape relative to patient support apparatus icon 152 as the actual active zone bears to the actual patient support apparatus 20.

In the example shown in FIG. 18, controller 70 is configured to display a plurality of successive center of gravity calculations of the patient's position. These center of gravity positions are displayed via icons 208, 208a-e shown on patient support apparatus icon 152. Position icon 208 displays the most recently calculated center of gravity of the patient. Position icon 208a displays the second most recently calculated center of gravity of the patient. Position icon 208b displays the third most recently calculated center of gravity of the patient. The remaining icons 208c-e show the fourth through sixth most recently calculated center of gravity of the patient, respectively. In some embodiments, controller 70 is configured to display the location icons 208 in a manner in which the oldest calculation of the center of gravity (e.g. location icon 208e in FIG. 18) is the most faded, while the most recent calculation of the patient's center of gravity (e.g. position icon 208 in FIG. 18) is the least faded.

In addition, controller 70 is configured to repetitively update the successive position icons 208 as the patient moves. Thus, the next time controller 70 calculates the patient's current center of gravity, controller 70 will delete the oldest position icon 208e from the location on patient support apparatus icon 152 shown in FIG. 17. Position icons 208 and 208a-d will remain in their currently shown places, and controller 70 will add new position icon 208 at the location corresponding to the most recent calculation of the patient's center of gravity. In this manner, controller 70 updates the location icons 208 so that the most recent calculation of the patient's center of gravity, as well as the five previous calculations of the patient's center of gravity, are shown on patient support apparatus icon 152 via location icons 208, 208a-e. It will, of course, be understood that controller 70 may be modified to display a different number of location icons (e.g. location icons 208 for less than the six most recent calculations of the patient's center of gravity, or location icons 208 for more than the six most recent calculations of the patient's center of gravity).

FIG. 19 illustrates a patient location screen 230 that may be displayed by controller 70 on display 60. Controller 70 may be configured to display patient location screen 230 as a standalone screen and/or it may be configured to integrate the content of screen 230 into one of the other screens shown herein (such as by replacing the patient support apparatus icon 152 and/or being superimposed upon the patient support apparatus icon 152 in any of the screens disclosed herein that include icon 152). Patient location screen 230, like exit detection information screen 210, displays a series of successive readings of the patient's center of gravity. Patient location screen 230 includes an X-axis 232, a Y-axis 234, a plurality of patient location icons 208, a direction indicator 236, and a line indicator 238. Controller 70 is configured to display the last five calculations of the patient's center of gravity on screen 230 through the use of patient location icons 208. Icon 208 corresponds to the most recent calculation of the patient's center of gravity; icon 208a corresponds to the second most recent calculation of the patient's center of gravity; icon 208b corresponds to the third most recent calculation of the patient's center of gravity; etc.

In addition to displaying a plurality of successive readings of the patient's center of gravity, controller 70 is configured to add a direction indicator 236 on screen 230. Direction indicator 236 shows the direction of movement of the five successive patient location icons 208. That is, direction indicator 236 has an arrow with its head positioned adjacent the most recent location icon 208, and pointed generally in the direction as an imaginary line drawn between the most recent location icon 208 and the second most recent location icon 208a. The tail end of direction indicator 236 is positioned adjacent the oldest patient location icon 208d and generally points in a direction similar to that of an imaginary line drawn from icon 208d to 208c. Direction indicator 236 therefore provide an easy to understand graphic indicator of the direction in which the patient has moved in the last moment. As with screen 210, controller 70 is configured to update the position of icons 208, as well as direction indicator 236, in real time in screen 230 as new calculations of the patient's center of gravity are made by controller 70.

Controller 70 also displays a line indicator 238 on screen 230 that extends between each of the successive calculations of the patient's center of gravity. Line indicator 238 extends between the icons 208 in a chronological manner. That is, a first end of the line starts at the oldest position icon 208d, moves to the second oldest position 208c, then to the third oldest position icon 208b, then to the fourth oldest position icon 208a, and finally ends at the most recent position icon 208. In some embodiments, line indicator 238 may include an arrow head at its end adjacent the most recent position icon 208. Alternatively, or additionally, controller 70 may be configured to display line indicator 238 with an arrow head, or some other kind of directional indicator, in lieu of direction indicator 236, or controller 70 may be configured to display direction indication 236 without displaying line indicator 238.

In some embodiments, controller 70 is configured to record the patient's center of gravity positions and store them in memory 72 with a time stamp. In such embodiments, screen 230 (and/or any of the other screens discussed herein) may include a slider bar, or other control, for allowing a user to view previous centers of gravity of the patient. In such embodiments, controller 70 may be configured to display the previous centers of gravity via a plurality of successive location icons 208 so that the user sees not only the prior centers of gravity, but also the general direction of the movement of the patient at the moment surrounding those center of gravity calculations.

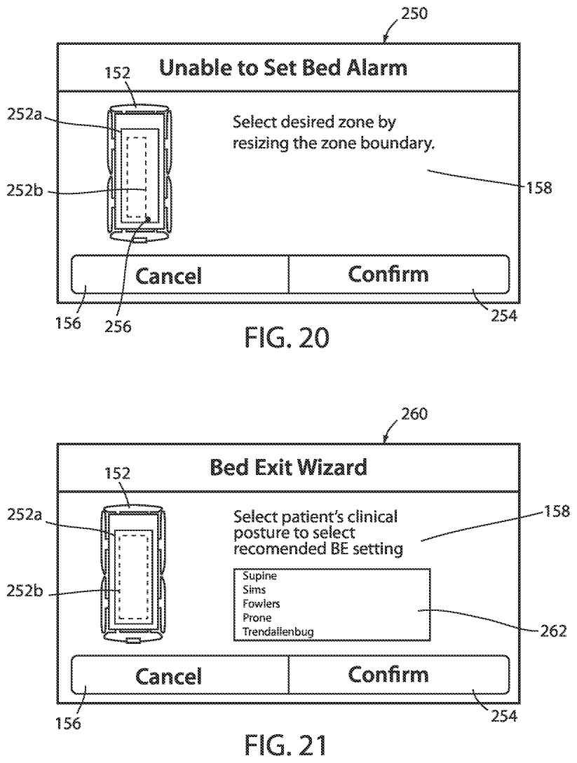

FIG. 20 illustrates an exit detection zone customization screen 250 that controller 70 may display on display 60 in response to a user pressing, for example, control 58a (FIG. 4) of patient support apparatus 20. Alternative methods for navigating to screen 250 may also, or alternatively, be implemented. Exit detection zone customization screen 250 includes a message area 158, a confirm control 254, a cancel control 156, and a first exit detection zone icon 252a and a second exit detection zone icon 252b that are both positioned on top of patient support apparatus icon 152. Second exit detection zone icon 252b includes a selection point 256 that allows a user to drag and re-size second exit detection zone icon 252b. As shown in message area 158, exit detection zone customization screen 250 is provided in order to allow a user to select an exit detection zone icons 252a and/or 252b and resize the boundary to a customized size, shape, and/or position on patient support apparatus 20. After the user selects the confirm control 254, the re-sized, reshaped, and/or repositioned boundary of icons 252a and/or 252b is then used by controller 70 for determining whether to issue an exit alert or not. That is, controller 70 thereafter compares the center of gravity of the patient to the resized, reshaped, and/or repositioned boundary of the selected icon 252a or 252b and, if the patient's center of gravity is outside of that boundary, controller 70 issues an exit detection alert.

Controller 70 is therefore configured to translate the icons 252a and/or 252b into proportionally sized and positioned exit detection zones that are used for determining whether to issue an exit detection alert. In other words, the size, shape, and position of a particular exit detection zone icon 252a or 252b relative to patient support apparatus icon 152 is used by controller 70 to define an actual exit detection zone that bears the same size, shape, and position relative to the actual patient support apparatus 20.

In some embodiments, controller 70 is configured to allow the user to adjust the size, shape, and/or position of an exit detection icon by selecting and dragging selection point 256. In such embodiments, movement of the selection point 256 causes controller 70 to behave in a manner similar to conventional windows in conventional software programs. In some embodiments, double pressing on selection point 256 causes an additional menu to be displayed that allows the user to change the shape of the exit detection zone icon 252, and/or to use other tools for editing the exit detection zone icon 252. Exit detection zone customization screen 250 therefore lets the user custom define the boundaries, shape, and/or position of the exit detection zone(s) used by the exit detection system.

In some embodiments, the user is able to save exit detection zone icons 252 that are customized using screen 250. The user may also assign a name to the saved zone icons, and controller 70 may be configured to display the saved zone icons-along with their name-on an exit detection zone selection screen, such as screen 250, so that the user is able to select exit detection zones that were previously customized.

FIG. 21 illustrates an exit detection zone selection screen 260 that controller 70 may display on display 60 in response to a user pressing, for example, control 58a (FIG. 4) of patient support apparatus 20, and/or in response to a user pressing a corresponding control displayed on another screen. Alternative methods for navigating to screen 260 may also, or alternatively, be implemented. Exit detection zone selection screen 260 includes a message area 158, a confirm control 254, a cancel control 156, a first exit detection zone icon 252a, a second exit detection zone icon 252b, a patient support apparatus icon 152, and a listing 262 of clinical configurations for patient support apparatus 20. Screen 260 allows a user to select one the clinical configurations listed in listing 262, and controller 70 is configured to automatically implement an exit detection zone having a size, shape, and position that are tailored to the particular clinical configuration selected by the user.

As shown in FIG. 21, listing 262 includes a supine position, a Sims' position, a Fowler's position, a prone position, a Trendelenburg position, a reverse Trendelenburg position, etc. After the uses selects one of the positions listed in listing 262, controller 70 is configured to select an exit detection zone 252 that has a size, shape, and position that is specifically tailored for the selected position. Thus, for example, if the user selects the Fowler's position, which corresponds to the Fowler section being raised to between about forty-five degrees to about sixty degrees, the patient's center of gravity will be shifted toward the foot end of patient support apparatus 20, as compared to when the patient is in a prone position, or when the Fowler section is lowered to a flat, or nearly flat, orientation. Accordingly, the zone 252 that is specially tailored for the Fowler's position will be shifted toward the foot end.

In some embodiments, after the user selects a particular position in the listing 262, controller 70 is configured to select a plurality of zones 252 of different size, shape, and/or location that correspond to the particular position selected from listing 262. The plurality of zones allows the user to further select the sensitivity level for the exit detection alert. Thus, for example, if the user selects the prone position, controller 70 is configured, at least in some embodiments, to allow the user to further select a low sensitivity level for the prone position, a medium sensitivity level for the prone position, and/or a high sensitivity level for the prone position. Typically, although not necessarily, the different sensitivity levels correspond to zones 252 that have different sizes, and therefore allow the user to move to different degrees before triggering an exit alert.

In some embodiments, the shape of the zones used by controller 70 when carrying out the exit detection function may be varied from the rectangles shown herein (e.g. icons 204, 222, 252). In addition, the size and/or shape of the zones used by controller may be dependent upon, and be dynamically adjusted in response to, the current position of one or more components of patient support apparatus 20, such as, but not limited to, the up/down status of one or more of the siderails 36, the angle of head section 42, the height of litter frame 28, etc. Still other factors may influence and/or cause changes to the size and/or shape of the zones used when carrying out the exit detection function.

In some embodiments, controller 70 is configured to transmit the center of gravity readings it takes to patient support apparatus 20 for forwarding to one or more mobile electronic devices 86 (FIG. 5). In such embodiments, patient support apparatus server 82 and/or caregiver assistance application 84 may be configured to cause the mobile electronic device 86 to display on display 88 any of the information shown in any of the screens shown herein in FIGS. 6-21 (or the screens themselves), thereby enabling a remotely positioned user to see the patient's center of gravity (including its movement) and/or to see the progress of the force sensor 54 calibration process. In some embodiments, the mobile electronic device 86 and/or caregiver assistance application 84 are adapted to allow the mobile electronic device 86 to send commands to the patient support apparatus 20 to remotely control one or more of the aspects of the patient support apparatus 20. For example, in some embodiments, mobile electronic device 86 is adapted to allow a user to remotely arm the exit detection function of patient support apparatus 20, remotely customize the size, shape, and/or location of an exit detection zone, and/or remotely select a position from listing 262 for use by the exit detection system. Mobile electronic device 86 may be configured to display screens like 200, 200a on display 88 in the event the patient is not correctly positioned on the patient support apparatus during the arming process.

It will be understood that the various icons shown herein may be changed in a variety of different manners from what is shown in the accompanying drawings. As but one example, one or more of the current patient position icons 208 can be modified to include the outline of a human with the center of the human shown at the location of the patient's center of gravity. A dot, or other indicator, may be incorporated into the human outline to visually inform the user of the patient's center of gravity relative to the patient support apparatus icon 152 and/or the rest of the human outline. Still other variations in the icons discussed herein may be implemented.

Additionally, or alternatively, controller 70 may be configured to display additional icons or indicators on any one or more of the screens disclosed herein. For example, in some embodiments, controller 70 may show a guided line (which may include an arrow head) that extends between initial patient position icon 206 and the current patient position icon 208 (see, e.g. FIGS. 16 and 17). The line may be similar to line indicator 238 of FIG. 19, and it may include an arrow head, or other direction indicator, to show the direction of movement of the patient.

It will be understood that the concepts disclosed herein may all be combined together in a patient support apparatus 20, or they may be individually integrated into a patient support apparatus 20. For example, in some embodiments, patient support apparatus 20 includes all the features discussed herein relating to the calibration process (and associated screens), but does not include any of the exit detection screens 200 or 200a or exit detection screens 210, 230. Alternatively, in some embodiments, patient support apparatus 20 includes one or more of the screens 200, 200a, 210, 230, 250, and/or 260 but does not include any or all of the force sensor calibration functions and/or their associated screens disclosed herein. In still other embodiments, still other combinations of the screens and functions disclosed herein may be integrated into a patient support apparatus 20.

It will also be understood that controller 70, in at least some embodiments, may be configured to automatically remove the effects of objects added to, or removed from, patient support apparatus 20 when it determines the patient's center of gravity and/or the patient's weight. In such embodiments, controller 70 automatically detects when an object is added or removed from patient support apparatus 20, enters or deletes the weight of the object in its weight log, and adjusts the weight readings from the force sensors 54 in a manner that removes the object's weight from the location calculations and weight readings described herein. In some embodiments, this automatic detection of the addition and/or removal of objects from patient support apparatus 20 is carried out in any of the manners disclosed in the following commonly assigned U.S. patent references: U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. patent 11,33,233 issued to Michael Hayes et al. on Jun. 15, 2021, and entitled PATIENT SUPPORT APPARATUS WITH PATIENT INFORMATION SENSORS; and U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG, the complete disclosures of all of which have already been incorporated herein by reference.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a support surface adapted to support a patient thereon;
a plurality of force sensors adapted to detect downward forces exerted on the support surface;
a control panel including an exit detection arming control adapted to arm an exit detection function;
a display; and
a controller adapted to calculate a center of gravity of the patient and, in response to a caregiver activating the exit detection arming control, determine whether the patient's center of gravity is inside or outside of a zone, and wherein if the controller determines that the patient's center of gravity is outside of the zone, the controller is further adapted to perform the following: (1) display a patient support apparatus icon; (2) display a first indicator at a first location on the patient support apparatus icon, the first location corresponding to the patient's center of gravity; and (3) display a target on the patient support apparatus icon, the target indicating where the patient's center of gravity should be in order for the controller to successfully arm the exit detection function.

2. The patient support apparatus of claim 1 wherein the controller is further adapted to display an arrow on the display indicating a direction in which the patient's center of gravity should move to get to the target.

3. The patient support apparatus of claim 1 wherein the first location corresponds to the patient's center of gravity at a time when the caregiver activates the exit detection arming control, and wherein the controller is further adapted to display a second indicator at a second location on the patient support apparatus icon, the second location corresponding to the patient's current center of gravity.

4. The patient support apparatus of claim 3 wherein the controller is further adapted to update the second location as the patient's center of gravity moves.

5. The patient support apparatus of claim 1 wherein the target is displayed as a rectangle icon on the patient support apparatus corresponding to the zone in which the patient's center of gravity must be located in order to arm the exit detection function.

6. The patient support apparatus of claim 5 wherein the controller is further adapted to change a characteristic of the rectangle icon when the patient's current center of gravity moves inside the zone.

7. The patient support apparatus of claim 6 wherein the characteristic is a color of the rectangle icon.

8. The patient support apparatus of claim 1 wherein the controller is adapted to arm the exit detection function after the patient's center of gravity moves inside the zone.

9. The patient support apparatus of claim 8 wherein the controller is further adapted, after arming the exit detection function, to repetitively calculate the patient's center of gravity and to display a plurality of icons on the patient support apparatus icon, the plurality of icons indicating successive calculations of the patient's center of gravity.

10. The patient support apparatus of claim 9 wherein the controller is further adapted to display a direction in which the successive calculations of the patient's center of gravity are moving.

11. The patient support apparatus of claim 1 wherein the controller is further adapted to display a calibration screen on the display, and wherein the calibration screen is adapted to guide a user through a calibration process for calibrating the plurality of force sensors.

12. The patient support apparatus of claim 11 wherein the calibration screen includes a first icon indicating a calibration location where the user should place a known weight on the patient support apparatus.

13. The patient support apparatus of claim 12 wherein the controller is further adapted to change a characteristic of the first icon after the user has placed the known weight on the patient support apparatus at the calibration location and the controller has taken a set of readings from the plurality of force sensors.

14. The patient support apparatus of claim 11 wherein the calibration screen includes a plurality of location icons indicating a plurality of calibration locations where the user should place a known weight on the patient support apparatus.

15.-54. (canceled)

55. A patient support apparatus comprising:
a support surface adapted to support a patient thereon;
a plurality of force sensors adapted to detect downward forces exerted on the support surface;
a control panel including an exit detection arming control adapted to arm an exit detection function;
a display; and
a controller adapted to repetitively calculate the patient's center of gravity using outputs from the plurality of force sensors, to compare the patient's center of gravity to a zone, to issue an exit alert if the patient's center of gravity moves outside of the zone, and to display an exit detection customization screen on the display, the exit detection screen being adapted to allow the caregiver to graphically change a size of a zone icon displayed therein, and wherein the controller is further adapted to change a size of the zone in a manner that corresponds to the size of the zone icon.

56. The patient support apparatus of claim 55 wherein the controller is further adapted to display an exit detection selection screen on the display, the exit detection selection screen being adapted to allow the caregiver to select from a list of predefined zones that correspond to different configurations of the patient support apparatus, and wherein the controller is further adapted to determine at least one of a size, shape, or position of the zone based on the predefined zone selected by the caregiver.

57. The patient support apparatus of claim 55 wherein the controller is further adapted to display a plurality of icons on the display, the plurality of icons indicating successive calculations of the patient's center of gravity.

58. The patient support apparatus of claim 57 wherein the controller is further adapted to display a direction in which the successive calculations of the patient's center of gravity are moving.

59. The patient support apparatus of claim 57 wherein the plurality of icons includes at least three icons displayed simultaneously on the display.

60. The patient support apparatus of claim 57 wherein the controller is adapted to display a calibration screen on the display, wherein the calibration screen is adapted to guide a user through a calibration process for calibrating the plurality of force sensors, and the calibration screen includes a patient support apparatus icon and a first location icon on the patient support apparatus icon indicating a first calibration location where the user should place a known weight on the patient support apparatus, and wherein the controller is further adapted to provide a first notification on the display after user has placed the known weight on the patient support apparatus at the first calibration location and the controller has taken a first set of readings from the plurality of force sensors.

61.-76. (canceled)
